(19) United States
(12) Plant Patent Application Publication
(10) Pub. No.: US 2019/0090398 P1
(43) Pub. Date: Mar. 21, 2019

(54) Gaultheria plant named `Hokutotakaji-001'

(50) ;

(71) Applicant: Tsukasa Tsuiki, Nanae (JP)

(72) Inventor: Tsukasa Tsuiki, Nanae (JP)

(21) Appl. No.: 15/732,116

(22) Filed: Sep. 20, 2017

(51) Int. Cl. A01H 6/00

(52) U.S. Cl. PLT/226

(57) ABSTRACT

A new cultivar of Gaultheria procumbens plant named ‘Hokutotakaji-001’ that is characterized by its compact, low-growing and weeping plant habit, glossy dark green and bronze colored foliage, clusters downward-facing bell-shaped white flowers held on orange-red stems and white berries.

Genus and species: Gaultheria procumbens.

Variety denomination: ‘Hokutotakaji-001’.

### BACKGROUND OF THE NEW PLANT

The present invention relates to a new and distinct cultivar of Gaultheria, commonly known as Wintergreen, which is grown as an ornamental plant for use in containers and in the landscape. The new cultivar is known botanically as Gaultheria procumbens and will be hereinafter referred to by the denomination ‘Hokutotakaji-001’.

During December 2009 to January 2010, the inventor observed within his crop of red-berried Gaultheria procumbens (species, unpatented) a naturally occurring whole-plant mutation which bore white-colored berries. The inventor also observed that the whole plant mutation exhibited additional desirable characteristics including large berries, weeping plant habit and smaller leaves which allow the white berries to be more visible.

The inventor first propagated ‘Hokutotakaji-001’ in June 2011 using the method of stem cuttings. The inventor has confirmed that ‘Hokutotakaji-001’ is stable and reproduces true to type in each successive generation of asexual reproduction.

### CROSS-REFERENCE TO RELATED APPLICATIONS

A Plant Breeders Rights application was filed in Japan, application number 29854, on Jan. 9, 2015. The present variety has not been sold or made publicly available more than one year prior to the filing date of the instant application.

### SUMMARY

The following traits have been repeatedly observed and represent the characteristics of the new Gaultheria cultivar ‘Hokutotakaji-001’. These traits in combination distinguish ‘Hokutotakaji-001’ from all other varieties of Gaultheria known to the inventor. ‘Hokutotakaji-001’ has not been tested under all possible conditions and phenotypic differences may be observed with variations in environmental, climatic and cultural conditions.

1. ‘Hokutotakaji-001’ exhibits a compact, low-growing and weeping plant habit.

2. ‘Hokutotakaji-001’ exhibits glossy dark green foliage.

3. From around October to March, the emerging leaves of ‘Hokutotakaji-001’ develop a bronzed appearance.

4. In June and July, ‘Hokutotakaji-001’ exhibits clusters of downward-facing bell-shaped white-colored flowers which are held on orange-red stems.

5. From fall until spring (i.e. throughout winter) ‘Hokutotakaji-001’ exhibits clusters of downward-facing round white berries held on orange-red stems.

6. The berries of ‘Hokutotakaji-001’ can achieve a diameter of 20 mm.

7. After 1 year of growth in a 9 cm container, a plant of ‘Hokutotakaji-001’ is 10 cm in height and 15 cm in width.

8. After 2 years of growth in a 13 cm container, a plant of ‘Hokutotakaji-001’ is 14 cm in height and 33 cm in width.

### DESCRIPTION OF THE PHOTOGRAPHS

The accompanying color drawings illustrate the overall appearance of the plant and flowers of the new Gaultheria variety named ‘Hokutotakaji-001’. The drawings have been made from digital photographs of plants of ‘Hokutotakaji-001’, which were grown in the inventor's unheated greenhouse in Nanae-town, Hokkaido, Japan.

FIG. 1 depicts a two-year old plant of ‘Hokutotakaji-001’ in early summer and illustrates the downward-facing white flowers and the bronzed coloration of recently-emerged leaves.

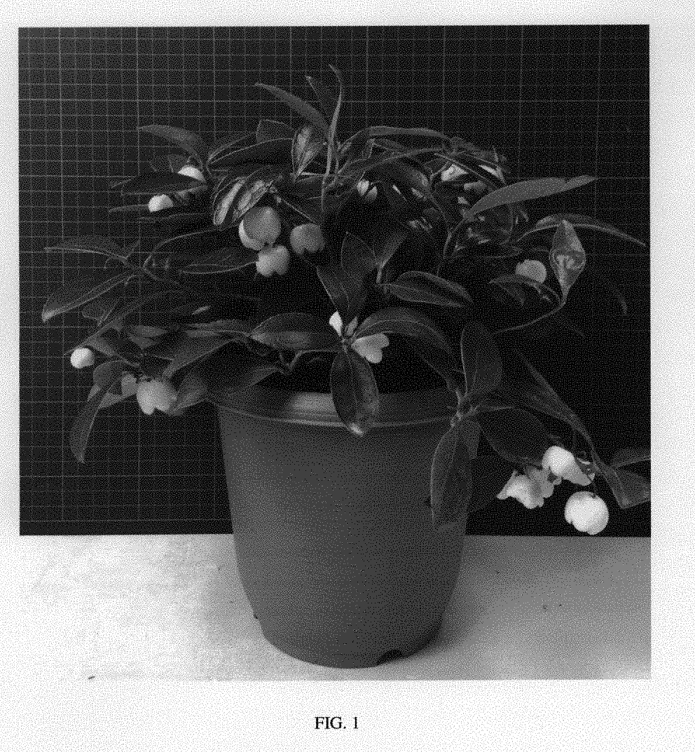

FIG. 2 depicts a close-up view of the dark green and bronze colored foliage together with the last flowers and the first fruits (berries) of the season.

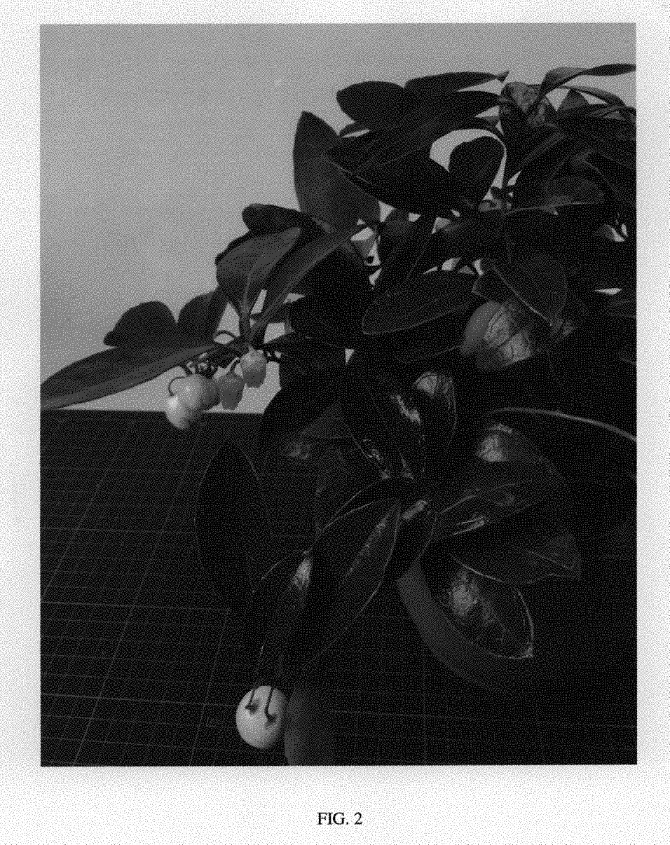

### DESCRIPTION OF THE NEW VARIETY

The following is a detailed description of Gaultheria procumbens ‘Hokutotakaji-001’ which has been grown and observed by the inventor in Hokkaido, Japan. The observed plants were two years old.

The color determinations are in accordance with the 2007 edition of the Colour Chart of The Royal Horticultural Society London, England, except where color terms of ordinary dictionary significance are used. Growing conditions, diseases and pests are similar to that of other Gaultheria.
Plant description:
   Botanical classification.—Gaultheria procumbens ‘Hokutotakaji-001’.
   Commercial classification.—Hardy groundcover shrub.
   Use.—For use in containers, and in the landscape as a border plant or as a groundcover.
   Parentage.—Selected as a white-berried seedling from red-berried seedlings of the species Gaultheria procumbens.
   Bloom period.—June and July.
   Plant habit.—Clumping, low growing, weeping.
   Vigor.—Moderate.
   Dimensions.—Height: 14 cm. Width: 33 cm.
   Hardiness.—The typical hardiness of the species is USDA Zone 3. ‘Hokutotakaji-001’ has not been tested in temperatures below USDA Zone 5.
   Propagation method.—Internodal stem cuttings with leaves attached.
   Root system.—Fibrous.
   Cultural requirements.—‘Hokutotakaji-001’ prefers ericaceous potting medium and planting soils. Light shade to full shade preferred.
   Growing time.—‘Hokutotakaji-001’ may be sold as a one-year old plant with occasional flowers and berries. A two-year old plant of ‘Hokutotakaji-001’ will carry numerous flowers and berries.
   Pests and diseases.—No pests or diseases have been observed to affect ‘Hokutotakaji-001’.
   Branches (quantity).—From 15 to 18 basal and lateral branches.
Stem:
   Stem color.—182D.
   Stem length.—70 mm to 80 mm.
   Stem diameter.—3 mm.
   Internode distance.—5 mm to 10 mm.
Foliage:
   Quantity (leaves on one stem).—8 to 10.
   Leaf length.—40 mm.
   Leaf width.—25 mm.
   Leaf color (green leaves, upper surface).—139A to 139B.
   Leaf color (green leaves, lower surface).—Ranges between 146C and 152D.
   Leaf color (bronze leaves, upper surface).—59A.
   Leaf color (bronze leaves, lower surface).—180B.
   Leaf shape.—Elliptic.
   Leaf base.—Attenuate.
   Leaf apex.—Acute.
   Leaf margin.—Senate.
   Leaf surface.—Smooth, glossy (upper surface).
   Leaf surface.—Matte (lower surface).
   Leaf veins color (green leaves, both surfaces).—146D.
   Leaf veins color (bronze leaves, both surfaces).—185C.
   Leaf fragrance.—Wintergreen mint fragrance when crushed.
Petioles:
   Petiole shape.—Round.
   Petiole length.—7 mm to 8 mm.
   Petiole width.—2 mm.
   Petiole color.—183D.
   Petiole surface.—Smooth, glossy.
Inflorescence:
   Inflorescence type.—Panicle.
   Inflorescence length (height).—25 mm to 35 mm.
   Inflorescence width.—15 mm to 20 mm.
   Number of inflorescences in one plant.—8 to 10.
   Number of flowers in one inflorescence.—Up to 5.
   Inflorescence fragrance.—None.
Flower buds:
   Flower buds, shape.—Obovate.
   Flower buds, length.—7 mm to 10 mm.
   Flower buds, diameter.—6 mm to 8 mm.
   Flower buds, color.—Ranges between NN155D and 155A.
Corolla, petals:
   Corolla shape.—Urceolate.
   Petal arrangement.—5 to 6 petals, fused longitudinally, apex free, recurving.
   Petal shape.—Oblanceolate.
   Petal length.—12 mm to 16 mm.
   Petal width.—6 mm to 8 mm.
   Petal color.—Ranges between NN155D and 155A.
   Petal surface.—Translucent, glossy.
Calyx, sepals:
   Calyx diameter.—8 mm to 10 mm.
   Calyx length (height).—3 mm to 4 mm.
   Sepal quantity.—5 to 6.
   Sepal shape.—Broadly triangular, apex acute.
   Sepal length.—4 mm to 5 mm.
   Sepal width.—3 mm.
   Sepal color.—NN155C.
   Sepal surface.—Smooth, slightly glossy. Opaque.
Peduncle:
   Peduncle length.—45 mm.
   Peduncle width.—1 mm to 2 mm.
   Peduncle color.—173A to 173B.
   Peduncle surface.—Puberulent.
Pedicel:
   Pedicel length.—16 mm.
   Pedicel diameter.—1.5 mm.
   Pedicel color.—34A changing abruptly to 162D towards calyx.
   Pedicel surface.—Puberulent.
Reproductive organs:
   Stamen.—10 to 12 in number.
   Filament length.—4 mm, diameter is less than 0.5 mm.
   Filament color.—NN155C.
   Anthers.—Oblong, 3 mm in length, dark brown.
   Pollen quantity.—Low.
   Pollen color.—N167B.
   Pistil quantity.—1.
   Stigma shape.—Truncate.
   Style length.—4 mm.
   Style color.—155C.
Fruit (berries):
   Berry quantity per branch.—Up to 5.
   Berry shape.—Globose, formed of 5 to 6 lobes, apex depressed and surrounded by deltoid lobe apices.
   Berry diameter.—Ranges between 11 mm and 20 mm.
   Berry color.—NN155D.
   Berry surface.—Glossy, glabrous.
Seed:
   Description.—Minute, irregular, color 21C.

### COMPARISON TO PARENTAL LINE AND TYPICAL VARIETIES

Whereas ‘Hokutotakaji-001’ bears white berries, the parent from which ‘Hokutotakaji-001’ was raised bears red-berries. The parent is itself a naturally-occurring whole-plant mutation of a plant of the red-berried species, Gaultheria procumbens.

Whereas ‘Hokutotakaji-001’ bears white berries, other varieties of Gaultheria known to the inventor bear red berries. In addition, in comparison with other varieties of Gaultheria known to the inventor, ‘Hokutotakaji-001’ exhibits some very large berries, a weeping habit and bears smaller leaves, which allow better visibility of the white berries.

I claim:

1. A new and distinct variety of Gaultheria plant named ‘Hokutotakaji-001’ as illustrated and described herein.
